(19) United States
(12) Patent Application Publication
(10) Pub. No.: US 2021/0244326 A1
(43) Pub. Date: Aug. 12, 2021

(54) SYSTEM, INFORMATION PROCESSING APPARATUS, AND INFORMATION PROCESSING METHOD

(71) Applicant: TOYOTA JIDOSHA KABUSHIKI KAISHA, Toyota-shi (JP)

(72) Inventors: Kazuya NISHIMURA, Anjo-shi (JP); Shin SAKURADA, Toyota-shi (JP); Hikaru GOTOH, Nagoya-shi (JP)

(73) Assignee: TOYOTA JIDOSHA KABUSHIKI KAISHA, Toyota-shi (JP)

(21) Appl. No.: 17/169,747

(22) Filed: Feb. 8, 2021

(30) Foreign Application Priority Data
Feb. 12, 2020 (JP) 2020-021616

(51) Int. Cl. A61B 5/18; G06K 9/00; G09B 19/16; B60W 40/09; B60W 40/04; A61B 5/16

(52) U.S. Cl. CPC A61B 5/18; G06K 9/00805; G09B 19/167; A61B 2560/0242; B60W 40/04; A61B 5/162; B60W 40/09

(57) ABSTRACT

The present disclosure is to evaluate a driving ability of a driver. A system includes a vehicle and an information processing apparatus. The vehicle detects rush-out of a moving object with a possibility of colliding with the vehicle by a sensor mounted on the vehicle, and also detects that a driver of the vehicle takes an evasive operation for avoiding a collision with the moving object. The information processing apparatus obtains a first time point, which is a point in time at which the vehicle detects the rush-out of the moving object, and a second time point, which is a point in time at which the vehicle detects that the driver takes an evasive action, calculates a reaction time that is a time difference between the first time point and the second time point, and evaluates the driving ability of the driver based on the reaction time.

### CROSS REFERENCE TO THE RELATED APPLICATION

This application claims the benefit of Japanese Patent Application No. 2020-021616, filed on Feb. 12, 2020, which is hereby incorporated by reference herein in its entirety.

### BACKGROUND

### Technical Field

The present disclosure relates to a system, an information processing apparatus, and an information processing method, for evaluating the driving ability of a driver.

### Description of the Related Art

Patent literature 1 discloses a dementia risk determination system. The determination system in patent literature 1 includes a road information obtaining unit for obtaining road information, a vehicle information obtaining unit mounted on a vehicle for obtaining travel information of the vehicle, a driver information detection unit for detecting information of a driver of the vehicle, a situation determination unit for determining a driving situation of the vehicle based on the road information and the travel information, a violation determination unit for determining whether or not the driving situation determined by the situation determination unit falls under a predetermined traffic violation, and a risk determination unit for determining whether or not the driver has a dementia risk in cases where the violation determination unit determines that the driving situation falls under the traffic violation, and an output unit for outputting information about the dementia risk in cases where the driver has a dementia risk.

### CITATION LIST

### Patent Literature

Patent Literature 1: Japanese Patent Application Laid-Open Publication No. 2019-124975

### SUMMARY

The present disclosure has for its object to evaluate the driving ability of a driver of a vehicle.

### Solution to Problem

A system according to a first aspect of the present disclosure includes a vehicle and an information processing apparatus, wherein

the vehicle detects, by a sensor mounted on the vehicle, rush-out of a moving object that may collide with the vehicle, and detects that a driver of the vehicle has taken an evasive action for avoiding a collision with the moving object; and

the information processing apparatus obtains a first time point, which is a point in time at which the vehicle detects that the moving object has rushed out, and a second time point, which is a point in time at which the vehicle detects that the driver has taken an evasive action, calculates a reaction time that is a time difference between the first time point and the second time point, and evaluates the driving ability of the driver based on the reaction time.

An information processing apparatus according to a second aspect of the present disclosure includes a controller comprising at least one processor that performs:

obtaining, by a sensor mounted on a vehicle, a first time point, which is a point in time at which the vehicle has detected rush-out of a moving object that may collide with the vehicle, and a second time point, which is a point in time at which the vehicle has detected that a driver of the vehicle has taken an evasive action for avoiding a collision with the moving object;

calculating a reaction time that is a time difference between the first time point and the second time point; and

evaluating the driving ability of the driver based on the reaction time.

A method according to a third aspect of the present disclosure is an information processing method performed by a computer, the method comprising:

obtaining, by a sensor mounted on a vehicle, a first time point, which is a point in time at which the vehicle has detected rush-out of a moving object that may collide with the vehicle, and a second time point, which is a point in time at which the vehicle has detected that a driver of the vehicle has taken an evasive action for avoiding a collision with the moving object;

calculating a reaction time that is a time difference between the first time point and the second time point; and

evaluating the driving ability of the driver based on the reaction time.

### Advantageous Effects of the Invention

According to the present disclosure, it is possible to evaluate the driving ability of a driver.

### BRIEF DESCRIPTION OF THE DRAWINGS

FIG. 1 is a view illustrating a schematic configuration of an evaluation system;

FIG. 2 is a block diagram schematically illustrating an example of a hardware configuration of a vehicle according to a first embodiment;

FIG. 3 is a block diagram schematically illustrating an example of a hardware configuration of an in-vehicle device according to the first embodiment;

FIG. 4 is a flowchart of evaluation processing;

FIG. 5 is a block diagram schematically illustrating an example of a hardware configuration of an in-vehicle device according to a second embodiment;

FIG. 6 is a graphic representation of evaluation values stored in an evaluation information database;

FIG. 7 is a flowchart of reduction determination processing;

FIG. 8 is a block diagram schematically illustrating an example of a hardware configuration of a vehicle according to a third embodiment; and

FIG. 9 is a view illustrating an example of a table configuration of limitation information.

### DESCRIPTION OF THE EMBODIMENTS

A system according to a first aspect of the present disclosure is a system including a vehicle and an information processing apparatus. When the vehicle is traveling, a moving object may rush out against the vehicle. Here, as the moving object, there can be exemplified an object such as a pedestrian, a bicycle, another vehicle, an animal, a falling object or the like. Then, in cases where the moving object, which has rushed out against the vehicle, is likely to come into contact with the vehicle, the driver of the vehicle will take an evasive action for avoiding a collision with the moving object. In the system according to the first aspect of the present disclosure, the driving ability of the driver is evaluated on the basis of a period of time from when the moving object rushes out against the vehicle to when the driver performs the evasive action. Specifically, the vehicle detects, by a sensor mounted on the vehicle, the rush-out of the moving object that may collide with the vehicle. The vehicle also detects that the driver of the vehicle has taken an evasive action for avoiding a collision with the moving object. Then, the information processing apparatus obtains a first time point, which is a point in time at which the vehicle detected the rush-out of the moving object that might collide with the vehicle, and a second time point, which is a point in time at which the vehicle detected that the driver took an evasive action for avoiding a collision with the moving object. Then, the information processing apparatus calculates a reaction time that is a time difference between the first time point and the second time point. This reaction time is a period of time from when the moving object rushed out against the vehicle to when the driver performed the evasive action for avoiding a collision with the moving object.

Then, the information processing apparatus evaluates the driving ability of the driver based on the reaction time. Here, the driving ability of the driver is an ability of the driver related to cognition, determination (judgement) or operation, which is necessary for safe driving of the vehicle. In this manner, according to the system of the first aspect of the present disclosure, it is possible to evaluate the driving ability of the driver based on a driving operation on the vehicle that is routinely performed by the driver without using a special device such as a driving simulator or the like.

Hereinafter, specific embodiments of the present disclosure will be described with reference to the drawings. The dimensions, materials, shapes, relative arrangements, and the like of the component parts or elements described in the embodiments are not intended to limit the technical scope of the present disclosure to only those unless otherwise specified.

### First Embodiment

(Outline of System)

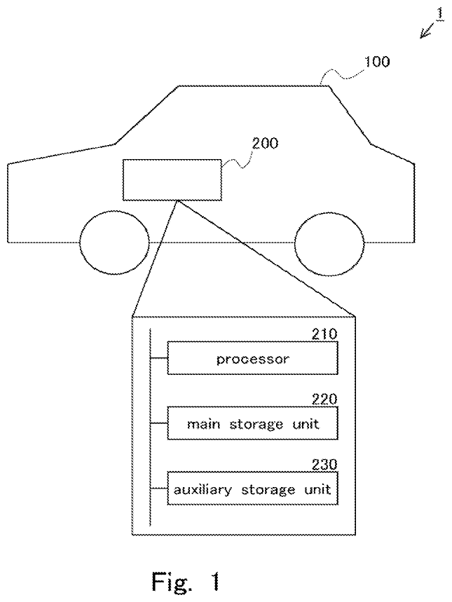

An evaluation system 1 in a first embodiment will be described with reference to FIG. 1. FIG. 1 is a view illustrating a schematic configuration of the evaluation system 1. The evaluation system 1 is configured to include a vehicle 100 and an in-vehicle device 200 mounted on the vehicle 100.

When the vehicle 100 is traveling, a moving object may rush out against the vehicle 100. Here, as the moving object, there can be exemplified an object, such as a pedestrian, a bicycle, another vehicle, an animal, a falling object or the like. Then, in cases where the moving object, which has rushed out against the vehicle 100, is likely to come into contact with the vehicle 100, the driver of the vehicle 100 will take an evasive action for avoiding a collision with the moving object. Here, as the evasive action taken by the driver, there can be mentioned application of a brake or turning of a steering wheel.

The vehicle 100 detects, by means of a sensor mounted on the vehicle 100, the rush-out of a moving object that may collide with the vehicle 100. The vehicle 100 also detects that the driver has taken an evasive action. Then, the vehicle 100 transmits, to the in-vehicle device 200, first time point information indicating a first time point, which is a point in time at which the rush-out of the moving object was detected, and second time point information indicating a second time point, which is a point in time at which the evasive action of the driver was detected. Here, note that a method of detecting that the driver of the vehicle 100 has taken an evasive action will be described later.

Then, the in-vehicle device 200 calculates a reaction time that is a time difference between the first time point and the second time point. This reaction time is a period of time from when the moving object rushes out against the vehicle 100 to when the driver performs an operation for avoiding a collision with the moving object. At this time, it can be said that the longer the reaction time is, the lower the driving ability of the driver is. In other words, the shorter the reaction time is, the higher the driving ability of the driver is. Therefore, the in-vehicle device 200 evaluates the reaction ability of the driver based on the reaction time.

The in-vehicle device 200 is a device that evaluates the driving ability of the driver of the vehicle 100. The in-vehicle device 200 is configured to include a computer having a processor 210, a main storage unit 220, and an auxiliary storage unit 230. The processor 210 is, for example, a central processing unit (CPU) or a digital signal processor (DSP). The main storage unit 220 is, for example, a random access memory (RAM). The auxiliary storage unit 230 is, for example, a read only memory (ROM). In addition, the auxiliary storage unit 230 is, for example, a hard disk drive (HDD) or a disk recording medium such as a CD-ROM, a DVD disk, or a Blu-ray disk. Also, the auxiliary storage unit 230 may be a removable medium (portable storage medium). Here, as the removable medium, there is mentioned, for example, a USB memory or an SD card.

In the in-vehicle device 200, an operating system (OS), various kinds of programs, various kinds of information tables and the like are stored in the auxiliary storage unit 230. In addition, in the in-vehicle device 200, the processor 210 loads a program stored in the auxiliary storage unit 230 into the main storage unit 220 and executes the program, thereby implementing various kinds of functions which will be described later. However, some or all of the functions of the in-vehicle device 200 may be implemented by a hardware circuit such as an ASIC or an FPGA. Here, note that the in-vehicle device 200 is not necessarily implemented by a single physical configuration, but may be configured by a plurality of computers cooperating with each other. Also, note that in the present embodiment, the in-vehicle device 200 corresponds to an “information processing apparatus” according to the present disclosure.

(System Configuration)

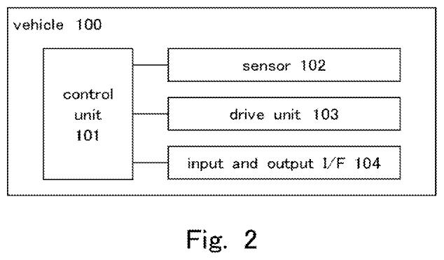

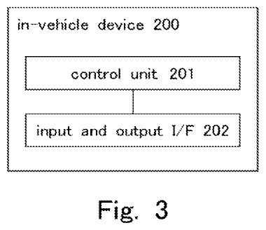

Next, the hardware configurations of the vehicle 100 and the in-vehicle device 200, which together constitute the evaluation system 1, will be described with reference to FIGS. 2 and 3.

(Vehicle)

FIG. 2 is a block diagram schematically illustrating an example of a hardware configuration of the vehicle 100 in the present embodiment. The vehicle 100 is configured to include a control unit 101, a sensor 102, a drive unit 103, and an input and output interface 104.

The sensor 102 is a device for sensing a surrounding situation around the vehicle 100. Specifically, the sensor 102 is configured to include a stereo camera, a laser scanner, a LIDAR, a radar, or the like. The information on the situation around the vehicle 100 obtained by the sensor 102 is transmitted to the control unit 101.

The drive unit 103 includes an engine or a motor as a prime mover, and mechanisms (i.e., an inverter, a brake, and a steering mechanism) for causing vehicle 100 to travel. The drive unit 103 causes the vehicle 100 to travel based on control information for controlling the drive unit 103, which is generated by the control unit 101. Here, the control information includes output information for controlling the throttle opening of the engine or the rotational speed (the number of revolutions per unit time) of the motor, braking information for controlling the braking force of the brake, steering information for controlling the steering angle of the steering mechanism, and the like.

The input and output interface (hereinafter sometimes referred to as an “input and output I/F”) 104 is an interface for sending and receiving various kinds of data between the vehicle 100 and the in-vehicle device 200. As the input and output I/F 104, there is exemplified a universal serial bus (USE) interface, a Bluetooth (registered trademark) interface, or the like.

The control unit 101 has a function of performing arithmetic processing for controlling the vehicle 100. The control unit 101 is configured to include a processor. The control unit 101 generates control information based on operation of the vehicle 100 by the driver. At this time, the control unit 101 generates output information, braking information, and steering information based on operations of an accelerator pedal, a brake pedal, and the steering wheel by the driver, respectively.

In addition, the control unit 101 detects the environment around the vehicle 100 based on the information about the situation around the vehicle 100 obtained by the sensor 102. For example, the control unit 101 detects a moving object present around the vehicle 100. The control unit 101 tracks the moving object thus detected. In this case, for example, the control unit 101 obtains the relative speed of the moving object from a difference between the coordinates of the moving object detected one step before and the current coordinates of the moving object. Then, the control unit 101 predicts the moving position of the moving object based on the relative speed of the moving object, and determines whether or not there is a possibility of collision of the moving object with the vehicle 100. That is, the control unit 101 detects a moving object for which the driver should take an evasive action. In this way, the control unit 101 detects the environment around the vehicle 100 thereby to detect the rush-out of the moving object that may collide with the vehicle 100. Then, in cases where the control unit 101 detects the rush-out of the moving object with a possibility of collision with the vehicle 100, the control unit 100 transmits first time point information to the in-vehicle device 200 via the input and output I/F 104. Here, note that a publicly known method can be adopted as a method by which the control unit 101 detects the rush-out of the moving object that may collide with the vehicle 100.

Moreover, when the driver of the vehicle 100 takes an evasive action, the control unit 101 generates braking information and steering information based on the evasive action. At this time, for example, when an amount of operation of the brake pedal in the braking information exceeds a predetermined threshold value, the control unit 101 detects that the driver has taken an evasive action. In addition, for example, when an amount of operation of the steering wheel per unit time in the steering information exceeds a predetermined threshold value, the control unit 101 detects that the driver has taken an evasive action. That is, the control unit 101 detects that the driver of the vehicle 100 has taken an evasive action, when the driver suddenly brakes or suddenly turns the steering wheel. Then, the control unit 101, upon detecting that the driver has taken an evasive action, transmits second time point information to the in-vehicle device 200 via the input and output I/F 104. Here, note that a publicly known method can be adopted as a method by which the control unit 101 detects that the driver of the vehicle 100 has taken an evasive action.

(In-Vehicle Device)

FIG. 3 is a block diagram schematically illustrating an example of a hardware configuration of the in-vehicle device 200 according to the present embodiment. The in-vehicle device 200 includes a control unit 201 and an input and output I/F 202. Because the input and output I/F 202 is the same interface as the input and output I/F 104 in the vehicle 100, the description thereof will be omitted.

The control unit 201 has a function of performing arithmetic processing for controlling the in-vehicle device 200. The control unit 201 can be realized by the processor 210. The control unit 201 receives the first time point information and the second time point information from the vehicle 100 via the input and output I/F 202. The control unit 201 calculates a reaction time, which is a time difference between the first time point and the second time point. Then, the control unit 201 associates a range of the reaction time with an evaluation of the driving ability, and determines the evaluation of the driving ability based on the range of the reaction time to which the calculated reaction time belongs. Here, the evaluation of the driving ability is classified, for example, from level 10 (high driving ability) to level 1 (low driving ability). In this way, the control unit 201 evaluates the driving ability of the driver of the vehicle 100. Here, note that the evaluation result of the driving ability of the driver is not limited to the level classification based on the reaction time. For example, the length of the reaction time itself may be the evaluation result of the driving ability of the driver.

(Evaluation Processing)

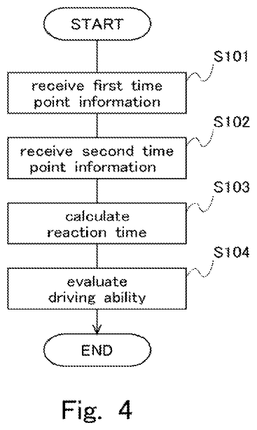

Next, the evaluation processing performed by the control unit 201 in the in-vehicle device 200 will be described with reference to FIG. 4. Here, the evaluation processing is a processing for evaluating the driving ability of the driver of the vehicle 100. FIG. 4 is a flowchart of the evaluation processing.

In the evaluation processing, first, in steps S101 and S102, first time point information and second time point information are received from the vehicle 100. Steps S101 and S102 may be executed simultaneously. Then, in step S103, a reaction time, which is a time difference between the first time point and the second time point, is calculated. In step S104, the driving ability of the driver of the vehicle 100 is evaluated based on the reaction time calculated in step S103.

As described above, according to the in-vehicle device 200 of the present embodiment, it is possible to evaluate the driving ability of the driver based on the driving operation of the vehicle 100 that is routinely performed by the driver of the vehicle 100, without using a special device such as a driving simulator or the like. In addition, it becomes possible to evaluate the driving ability of the driver based on the driving operation of the vehicle 100 that is routinely performed by the driver, thus making it possible to grasp the transition (or the change over time) of the driving ability of the driver. Therefore, in cases where the driving ability of the driver has been reduced or lowered due to, for example, a symptom such as aging, dementia or the like, it becomes possible to detect such a reduction in the driving ability.

Here, note that the control unit 201 in the in-vehicle device 200 may store the evaluation result of the driving ability of the driver of the vehicle 100 in the auxiliary storage unit 230 in association with a driver ID for identifying the driver. In this case, the evaluation result of the driver is stored in the auxiliary storage unit 230 each time the evaluation processing illustrated in FIG. 4 is executed, that is, each time the rush-out of the moving object with respect to the vehicle 100 and the evasive action taken by the driver are detected. Then, the control unit 201 may evaluate the driving ability of the driver based on a plurality of evaluation results of the driving ability of the same driver stored in the auxiliary storage unit 230. At this time, the control unit 201 may evaluate the driving ability of the driver based on, for example, the evaluation results of a predetermined number of times or evaluation results in a predetermined period of time. For example, the control unit 201 evaluates the driving ability of the driver of the vehicle 100 based on the average value or the most frequent value of a plurality of evaluation results of the driving ability.

In addition, the in-vehicle device 200 may be a component of the vehicle 100. That is, the in-vehicle device 200 and the vehicle 100 may be a single device, and the vehicle 100 may have the function of the in-vehicle device 200. In this case, the processing or the like executed by the control unit 201 in the in-vehicle device 200 is executed by the control unit 101 in the vehicle 100.

Moreover, in the present embodiment, the driving ability of the driver is evaluated by the in-vehicle device 200 mounted on the vehicle 100, but the driving ability of the driver may be evaluated by an external server. At this time, the vehicle 100 and the external server are connected to each other by a network. As the network, for example, a wide area network (WAN), which is a global public communication network such as the Internet, or a telephone communication network such as a mobile phone network may be adopted. The vehicle 100 transmits the first time point information and the second time point information to the external server via the network. Then, the external server evaluates the driving ability of the driver of the vehicle 100 based on the first time point information and the second time point information received. Thus, the driving ability of the driver of the vehicle 100 can also be evaluated by the external server. In this manner, by evaluating the driving ability of the driver by the external server, it is possible to grasp the transition of the driving ability of the driver, even in cases where the driver changes vehicles.

### Second Embodiment

In a second embodiment, the in-vehicle device 200 accumulates the evaluation results (hereinafter, sometimes referred to as “evaluation values”) of the driving ability of the driver of the vehicle 100, and determines whether or not the driving ability of the driver has reduced (deteriorated). Hereinafter, only differences of this second embodiment from the first embodiment will be described.

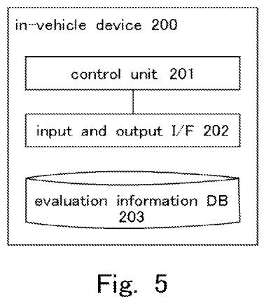

FIG. 5 is a block diagram schematically illustrating an example of the hardware configuration of the in-vehicle device 200 according to this second embodiment. In the second embodiment, the in-vehicle device 200 is configured to further include an evaluation information database (hereinafter, sometimes referred to as an “evaluation information DB”) 203. The evaluation information DB 203 is a database for storing evaluation values. The evaluation information DB 203 can be realized by the auxiliary storage unit 230. When the evaluation processing ends, the control unit 201 stores the evaluation values obtained by the evaluation processing in the evaluation information DB 203. The control unit 201 can grasp the transition of the evaluation values, by virtue of the evaluation values stored in the evaluation information DB 203.

Then, the control unit 201 determines, based on the transition of the evaluation values, whether or not the driving ability of the driver of the vehicle 100 has reduced. At this time, the control unit 201 sets an evaluation reference value of the driving ability of the driver based on the evaluation values stored in the evaluation information DB 203. Here, the evaluation reference value is set based on the evaluation values in a first predetermined period of time. The first predetermined period of time is, for example, a certain period of time after the in-vehicle device 200 starts evaluating the driving ability of the driver. Then, an average value of the evaluation values in the first predetermined period of time is set as the evaluation reference value. Here, note that the first predetermined period of time may be a period of time in which the evaluation reference value becomes the highest at the time of setting the evaluation reference value. In addition, the evaluation reference value may be a predetermined value. Here, note that the first predetermined period of time in this second embodiment corresponds to a “predetermined period of time” according to the present disclosure.

In addition, the control unit 201 calculates an average value of the evaluation values in a second predetermined period of time (hereinafter, sometimes referred to as an “evaluation average value”). The second predetermined period of time is a period of time after the first predetermined period of time. That is, it is a period of time after the evaluation reference value of the driving ability of the driver was set. Here, the second predetermined period of time is, for example, a period of time in which the rush-out of the moving object was detected a predetermined number of times by the vehicle 100.

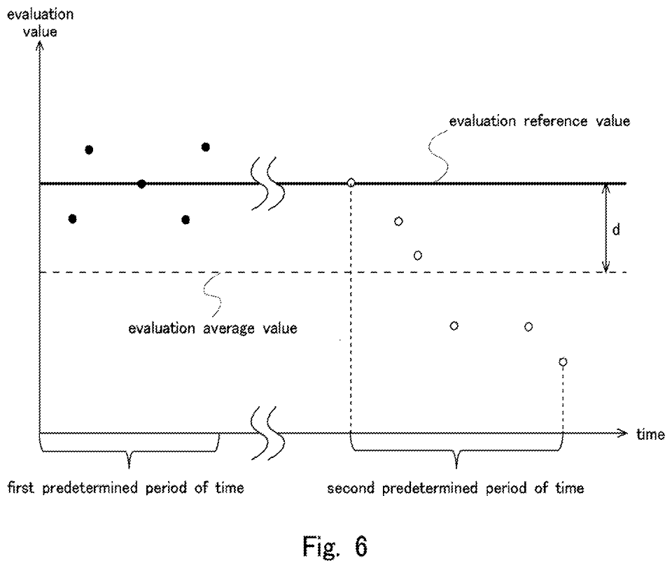

FIG. 6 is a graphic representation illustrating the evaluation values stored in the evaluation information DB 203. In FIG. 6, the horizontal axis represents time, and the vertical axis represents the evaluation values. In addition, the evaluation values in the first predetermined period of time are indicated by black circles. The evaluation values in the second predetermined period of time are indicated by white circles. Also, in FIG. 6, the evaluation reference value is indicated by a solid line. Moreover, the evaluation average value in the second predetermined period of time is indicated by a broken line. Here, the second predetermined period of time in FIG. 6 is set as a period of time in which the rush-out of moving objects has been detected six times by the vehicle 100. Then, when a difference d between the evaluation reference value and the evaluation average value is equal to or greater than a predetermined threshold value, the control unit 201 determines that the driving ability of the driver has reduced. Thus, in cases where the evaluation average value is lower than the evaluation reference value by a predetermined threshold value or more, the control unit 201 determines that the driving ability of the driver has reduced.

(Reduction Determination Processing)

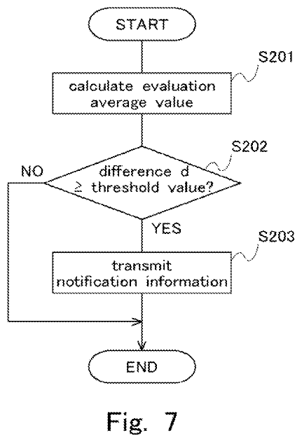

Now, reduction (or deterioration) determination processing executed by the control unit 201 in the in-vehicle device 200 will be described with reference to FIG. 7. Here, the reduction determination processing is a processing for determining whether or not the driving ability of the driver of the vehicle 100 has reduced. FIG. 7 is a flowchart of the reduction determination processing. The reduction determination processing is performed each time the second predetermined period of time elapses after the evaluation reference value was set.

In the reduction determination processing, first, in step S201, the evaluation average value in the second predetermined period of time is calculated based on the evaluation values stored in the evaluation information DB 203. Then, in step S202, it is determined whether or not the difference d between the evaluation reference value and the evaluation average value is larger than the predetermined threshold value. In cases where a negative determination is made in step S202, the reduction determination processing is terminated. At this time, it is determined that the driving ability of the driver has not reduced. On the other hand, in cases where an affirmative determination is made in step S202, it is determined that the driving ability of the driver has reduced. Then, in step S203, notification information for notifying the driver of the vehicle 100 of the fact that the driving ability of the driver has reduced is transmitted to the vehicle 100 via the input and output I/F 202. Thereafter, upon reception of the notification information, the vehicle 100 displays on a display unit of the vehicle 100 that the driving ability of the driver has reduced. In this way, the notification information allows the driver to grasp that the driving ability of the driver has reduced. Here, note that the in-vehicle device 200 may include a communication unit for radio or wireless communication, and may transmit the notification information to an information terminal carried by the driver. In addition, the in-vehicle device 200 may transmit the notification information to an information terminal possessed by a person who is related to the driver and registered in advance in the in-vehicle device 200. Here, the related person of the driver is a family or a friend of the driver or a third party related to the driver. In this way, by notifying the concerned person of the driver of the fact that the driving ability of the driver has reduced, the concerned person can grasp that the driving ability of the driver has reduced.

As described above, the control unit 201 can determine whether or not the driving ability of the driver of the vehicle 100 has reduced, based on the transition of the evaluation values obtained by the driving operation routinely performed by the driver. Thus, when the driving ability of the driver has been reduced due to a symptom such as aging or dementia, the reduction thereof can be detected.

Here, note that in the second embodiment, the evaluation reference value and the evaluation average value are an average value of the evaluation values in the first predetermined period of time, and an average value of the evaluation values in the second predetermined period of time, respectively. However, the evaluation reference value may be the most frequent value of the evaluation values in the first predetermined period of time. Also, the control unit 201 may determine the reduction in the driving ability of the driver, based on the most frequent value of the evaluation values in the second predetermined period of time, instead of the evaluation average value.

### Third Embodiment

In a third embodiment, in cases where it is determined in the reduction determination processing that the driving ability of the driver of the vehicle 100 has reduced, the in-vehicle device 200 transmits, to the vehicle 100, limitation information for setting a predetermined upper limit value for the acceleration of the vehicle 100 at the time when the driver steps on the accelerator pedal (hereinafter, sometimes simply referred to as “acceleration”). Hereinafter, only differences of this third embodiment from the second embodiment will be described.

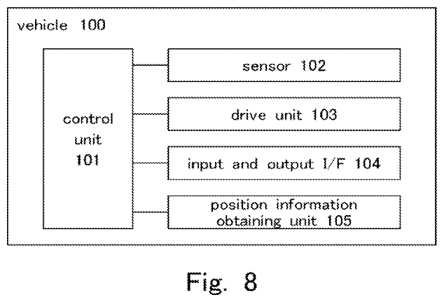

FIG. 8 is a block diagram schematically illustrating an example of a hardware configuration of the vehicle 100 in the third embodiment. The vehicle 100 is configured to further include a position information obtaining unit 105. The position information obtaining unit 105 is a device that obtains the current position of the vehicle 100. Specifically, the position information obtaining unit 105 is configured to include a GPS receiver or the like. The current position of the vehicle 100 obtained by the position information obtaining unit 105 is transmitted to the control unit 101 as position information.

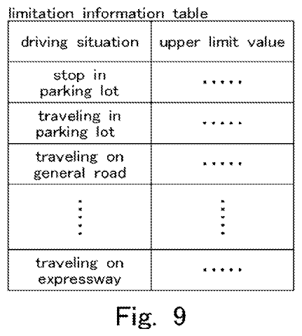

The control unit 201 in the in-vehicle device 200 transmits the limitation information to the vehicle 100 via the input and output I/F 202, in cases where it is determined in the reduction determination processing that the driving ability of the driver of the vehicle 100 has reduced. FIG. 9 is a view illustrating an example of a table configuration of the limitation information. As illustrated in FIG. 9, the limitation information table includes driving situation fields and upper limit value fields. In the driving situation fields, driving situations each representing a situation in which the vehicle is driving are entered, respectively. Here, as the driving situations, there can be mentioned some examples such as a situation in which the vehicle is stopped in a parking lot, a situation in which the vehicle is running in a parking lot, a situation in which the vehicle is traveling on an expressway, and a situation in which the vehicle is traveling on a general road, and the like.

In addition, an upper limit value of the acceleration of the vehicle 100 according to the driving situation of the vehicle 100 is entered in each of the upper limit value fields. At this time, for example, different upper limit values of the acceleration are entered, when the vehicle 100 is stopped in a parking lot and when the vehicle 100 is traveling on an expressway, respectively. That is, an upper limit value of the acceleration is set for each driving situation of the vehicle 100. For example, when the vehicle 100 is stopped in a parking lot, an upper limit value of acceleration is set so as to suppress a sudden start when the driver erroneously operates the accelerator pedal. Also, when the vehicle travels on an expressway, a certain amount of acceleration is required for the vehicle to merge into the main line of the highway. Therefore, the upper limit value of the acceleration at the time when the vehicle 100 is traveling on the expressway is set to be larger than the upper limit value of the acceleration when the vehicle 100 is stopped in the parking lot. Here, note that the upper limit value of the acceleration in the limitation information may be changed in accordance with the degree of reduction in the driving ability of the driver of the vehicle 100. For example, as the difference d between the reference value and the evaluation average value illustrated in FIG. 6 increases due to the reduction in the driver's driving ability, the upper limit value of the acceleration in the limitation information may be made smaller.

Upon receiving the limitation information from the in-vehicle device 200 via the input and output I/F 104, the control unit 101 in the vehicle 100 generates control information based on the limitation information. At this time, the control unit 101 can grasp the driving situation of the vehicle 100 based on the position information. For example, when the current position of the vehicle 100 in the position information is within a parking lot and the vehicle 100 is not moving, it is determined that the vehicle 100 is stopped in the parking lot. Then, the control unit 101 obtains the upper limit value of the acceleration corresponding to “stop in parking lot”. In this case, even if the driver of the vehicle 100 strongly depresses the accelerator pedal, the control unit 101 limits the throttle opening degree of the engine or the rotation speed (i.e., the number of revolutions per unit time) of the motor in the output information, based on the obtained upper limit value of the acceleration.

When the in-vehicle device 200 determines that the driving ability of the driver of the vehicle 100 has reduced, the acceleration of the vehicle 100 is limited. As a result, it is possible to prevent the vehicle 100 from suddenly starting due to an erroneous operation of the accelerator pedal by the driver whose driving ability has reduced. In addition, by determining the upper limit value of the acceleration based on the driving situation of the vehicle 100, it is possible to set a more appropriate limit of the acceleration in accordance with the driving situation of the vehicle 100.

### Other Embodiments

The above-described embodiments are merely some examples, and the present disclosure can be appropriately modified and implemented without departing from the spirit and scope of the present disclosure. In addition, the processing, means, devices, units, steps and the like described in the present disclosure can be freely combined and implemented as long as no technical contradiction occurs.

The processing described as being performed by one device or unit may be shared and performed by a plurality of devices or units. Alternatively, the processing described as being performed by different devices or units may be performed by one device or unit. In a computer system, a hardware configuration (server configuration) for realizing each function thereof can be changed in a flexible manner.

The present disclosure can also be achieved by supplying to a computer a computer program in which the functions described in the above embodiments are implemented, and causing one or more processors included in the computer to read and execute the program. Such a computer program may be provided to the computer by a non-transitory computer readable storage medium that can be connected to a system bus of the computer, or may be provided to the computer via a network. A non-transitory computer readable storage medium includes any type of medium suitable for storing electronic instructions or commands, such as, for example, any type of disk such as a magnetic disk (e.g., a floppy (registered trademark) disk or a hard disk drive (HDD)), an optical disk (e.g., a CD-ROM, a DVD disk, or a Blu-ray disk), a read-only memory (ROM), a random access memory (RAM), an EPROM, an EEPROM, a magnetic card, a flash memory, or an optical card.

What is claimed is:

1. A system including a vehicle and an information processing apparatus,
the vehicle being configured to detect, by a sensor mounted on the vehicle, rush-out of a moving object that may collide with the vehicle, and to detect that a driver of the vehicle has taken an evasive action for avoiding a collision with the moving object, and
the information processing apparatus being configured to perform:
obtaining a first time point, which is a point in time at which the vehicle detects that the moving object has rushed out, and a second time point, which is a point in time at which the vehicle detects that the driver has taken an evasive action;
calculating a reaction time that is a time difference between the first time point and the second time point; and
evaluating a driving ability of the driver based on the reaction time.

2. The system according to claim 1, wherein the information processing apparatus determines, based on a transition of an evaluation of the driving ability of the driver, whether or not the driving ability of the driver has reduced.

3. The system according to claim 2, wherein the information processing apparatus sets an evaluation reference value based on the evaluation of the driving ability of the driver in a predetermined period of time, and determines that the driving ability of the driver has reduced, when the evaluation of the driving ability of the driver after the predetermined period of time is lower than the evaluation reference value by a predetermined threshold value or more.

4. The system according to claim 3, wherein the information processing apparatus determines that the driving ability of the driver has reduced, when an average value or a most frequent value of the evaluation of the driving ability of the driver has reduced from the evaluation reference value by the predetermined threshold value or more after the predetermined period of time.

5. The system according to claim 2, wherein the information processing apparatus notifies the driver or a person related to the driver that the driving ability of the driver has reduced, when it is determined that the driving ability of the driver has reduced.

6. The system according to claim 2, wherein the information processing apparatus transmits, to the vehicle, limitation information for setting a predetermined upper limit value for acceleration of the vehicle, when it is determined that the driving ability of the driver has reduced.

7. The system according to claim 6, wherein in the limitation information, the predetermined upper limit value is set for each driving situation of the vehicle.

8. The system according to claim 1, wherein the vehicle detects that the driver of the vehicle has taken the evasive action, based on control information for controlling a drive unit of the vehicle.

9. An information processing apparatus including a controller comprising at least one processor configured to perform:
obtaining, by a sensor mounted on a vehicle, a first time point, which is a point in time at which the vehicle has detected rush-out of a moving object that may collide with the vehicle, and a second time point, which is a point in time at which the vehicle has detected that a driver of the vehicle has taken an evasive action for avoiding a collision with the moving object;
calculating a reaction time that is a time difference between the first time point and the second time point; and
evaluating a driving ability of the driver based on the reaction time.

10. The information processing apparatus according to claim 9, wherein the controller further performs determining whether or not the driving ability of the driver has reduced, based on a transition of an evaluation of the driving ability of the driver.

11. The information processing apparatus according to claim 10, wherein the controller sets an evaluation reference value based on an evaluation of the driving ability of the driver in a predetermined period of time; and the controller determines that the driving ability of the driver has reduced, when the evaluation of the driving ability of the driver after the predetermined period of time is lower than the evaluation reference value by a predetermined threshold value or more.

12. The information processing apparatus according to claim 11, wherein the controller determines that the driving ability of the driver has reduced, when an average value or a most frequent value of the evaluation of the driving ability of the driver is lower than the evaluation reference value by the predetermined threshold value or more after the predetermined period of time.

13. The information processing apparatus according to claim 10, wherein when the controller determines that the driving ability of the driver has reduced, the controller notifies the driver or a person related to the driver that the driving ability of the driver has reduced.

14. The information processing apparatus according to claim 10, wherein the controller transmits, to the vehicle, limitation information for setting a predetermined upper limit value for acceleration of the vehicle, when it is determined that the driving ability of the driver has reduced.

15. The information processing apparatus according to claim 14, wherein in the limitation information, the predetermined upper limit value is set for each driving situation of the vehicle.

16. An information processing method performed by a computer, the information processing method comprising:
obtaining, by a sensor mounted on a vehicle, a first time point, which is a point in time at which the vehicle has detected rush-out of a moving object that may collide with the vehicle, and a second time point, which is a point in time at which the vehicle has detected that a driver of the vehicle has taken an evasive action for avoiding a collision with the moving object;
calculating a reaction time that is a time difference between the first time point and the second time point; and
evaluating a driving ability of the driver based on the reaction time.

17. The information processing method according to claim 16, further comprising:
determining whether or not the driving ability of the driver has reduced, based on a transition of an evaluation of the driving ability of the driver.

18. The information processing method according to claim 17, further comprising:
setting an evaluation reference value based on an evaluation of the driving ability of the driver in a predetermined period of time; and determining that the driving ability of the driver has reduced, when the evaluation of the driving ability of the driver after the predetermined period of time is lower than the evaluation reference value by a predetermined threshold value or more.

19. The information processing method according to claim 17, further comprising:
notifying the driver or a person related to the driver that the driving ability of the driver has reduced, when it is determined that the driving ability of the driver has reduced.

20. The information processing method according to claim 17, further comprising: transmitting, to the vehicle, limitation information for setting a predetermined upper limit value for acceleration of the vehicle, when it is determined that the driving ability of the driver has reduced.
